(19) United States
(12) Patent Application Publication
(10) Pub. No.: US 2008/0267988 A1
(43) Pub. Date: Oct. 30, 2008

(54) Myelin specific IgE unencumbered by corresponding blocking antibodies as a causative factor in multiple sclerosis

(75) Inventor: Emanuel Calenoff, Dallas, TX (US)

Correspondence Address: BARNES & THORNBURG LLP, P.O. BOX 2786, CHICAGO, IL, 60690-2786, US

(73) Assignee: Enteron Limited Partnership, Oak Brook, IL (US)

(21) Appl. No.: 12/082,515

(22) Filed: Apr. 11, 2008

Related U.S. Application Data
(60) Provisional application No. 60/911,200, filed on Apr. 11, 2007.

(51) Int. Cl. A61K 39/00; G01N 33/53; G01N 33/567; C07K 16/42

(52) U.S. Cl. 424/185.1; 435/7.1; 435/7.21; 530/387.2

(57) ABSTRACT

Optimization and validation of a serum test confirms symptomatic autoimmune diseases e.g. multiple sclerosis (MS) or rheumatoid arthritis, and identifies early, silent diseases. The methods involve testing of subject sera for the presence of epitope-specific serum IgE and also non-IgE antibodies. The tests are immunoassays wherein serum autoantibodies complex with peptides structurally mimicking humoral, autoimmune epitopes on, for example, myelin basic protein (MBP), myelin oligodendrocyte glycoprotein (MOG) and proteolipid protein (PLP) for MS. Each peptide: (1) is 5-6 amino acids long; (2) structurally and functionally mimics the surface region of its parent protein; and (3) offers a correct fit for the antigen binding site of a single specific autoantibody. Relapse prediction tests and therapies employ the peptides.

### PRIORITY CLAIM

This application claims priority to copending U.S. provisional 60/911,200 filed Apr. 11, 2007.

### BACKGROUND

Screening tests for autoimmune diseases based upon quantification of epitope-specific, harmful serum autoantibody levels as a percentage of the total epitope-specific serum autoantibody level are described.

Examples of autoimmune diseases are multiple sclerosis (MS) and rheumatoid arthritis. Multiple sclerosis (MS) is an autoimmune disease, although agreement is lacking as to its instigating and sustaining causes. Some investigators postulate that MS is a T cell-mediated autoimmune disease. Other investigators have argued for humoral (antibody-mediated) autoimmunity. In either explanation, elucidation of the complete MS autoimmune process has not followed early enthusiasm generated by preliminary studies. Furthermore, recent investigation has cast doubt about the relevance of non-IgE antibodies to MS pathology.

MS is characterized by the proliferation of auto-reactive immune cells, their entry into the central nervous system (CNS) with subsequent perivascular inflammation and release of inflammatory cytokines, patchy demyelination and axonal injury or loss. Cell mediated aspects of immune injury have been emphasized as underlying MS at the expense of fully understanding the relevance of humoral (antibody-based) immune injury and whether one or the other plays the principal pathological role.

Although the potential role of antibody-mediated, immune dysfunction has been gaining increased interest in recent years, and anti-myelin IgG, and IgM, have been investigated, results have been conflicting, spotty, and ambiguous. Consideration of a potential disease-causing or effecting role of IgE autoantibodies has been missing possibly due to: (1) historical predisposition toward working with IgA, IgG, and IgM autoantibodies; (2) lack of practical experience in specific IgE quantification; (3) an unsubstantiated conviction that MS-related autoantibodies are produced in the perineural space and must be quantified in cerebrospinal fluid (CSF), not in serum; and (4) absence of a coherent explanation or theory as to overall MS pathology.

Serum anti-myelin IgE has not yet been adequately investigated despite reports that degranulating mast cells are present in active MS lesions (plaques). Neurologist-investigators have postulated rare or speculative mast cell activating processes to explain their role in lesion formation.

No explanation has been offered to bridge the understanding of how mast cells most commonly operate (i.e. allergy) and their potential role in MS. In atopic allergy, allergen-bound IgE is presented in dimer form, one IgE molecule separated from its partner by 40-100 angstroms, but both are sterically aligned in their Fc projection toward the mast cell membrane. The IgE dimer sterically fits onto waiting mast cell receptor pairs whereupon degranulation ensues. Degranulating mast cells expel lytic enzymes, histamine, and immune cell recruiting factors, leading to inflammation, tissue destruction, and blood brain barrier disruption.

A hallmark of rheumatoid arthritis is the significant, quantitative presence of mast cells indicating concomitant presence of collagen-specific IgE with all of its antigen-binding qualifications.

### SUMMARY

A plurality of target immunogenic peptides of a target protein wherein the target peptides produce a disease-specific immune response in a host, and wherein the target protein is causative of or associated with, a targeted disease, and wherein the peptides comprise the following structure:
(a) are 5-6 amino acids in length;
(b) have an amino acid sequence which is identical to a contiguous amino acid peptide region of a sequence of a protein designated the target protein;
(c) are characterized by a net hydrophilic structure of the peptide while on the protein surface;
(d) include two or more individually hydrophobic, constituent amino acids;
(e) display a net hydrophilicity value of 4.8 or less when the peptide is analyzed separately from its larger, parent protein sequence; and
(f) are characterized by an amino acid sequence that is structurally unique as to its amino acid sequence order.

The plurality of target immunogenic peptides are further characterized as having antigenic profiles which elicit an immune response specific for the target protein as determined by results of immunoassays of disease positive biological fluids compared to disease negative biological fluids.

A method for diagnosing a disease in a subject wherein a peptide is used as source antigen to quantify epitope-specific, potentially harmful antibody levels and levels of competing antibodies in a biological fluid obtained from the subject includes the steps of:
(a) dividing the harmful antibody levels by the level of the competing antibodies to derive a quotient value;
(b) comparing the quotient value of the subject to a quotient value range predetermined for a specific disease; and
(c) diagnosing the disease by assigning a positive or negative test result based upon the subject quotient value being higher or lower than a disease-positive threshold quotient value of the range.

The harmful antibody may be an IgE antibody, an IgA₂, IgG₁, IgG₃, or complement-fixing IgM antibody. The harmful antibody may also be an opsonizing antibody.

The competing antibody may be an IgA₁, IgG₂, IgG₄, or non-complement-fixing IgM antibody.

A method to screen for multiple sclerosis, includes the steps of:
(a) detecting myelin epitope-specific IgE antibodies to peptides serving as source antigens which structurally and fundamentally mimics humoral epitopes on the surface of myelin;
(b) ascertaining blocking antibodies by quantifying individual human kappa-chain-specific antibodies plus lambda chain-specific antibodies to the epitope-mimicking peptide and subtracting the quantity of the specific IgE, to reflect non-IgE specific antibody; and
(c) computing a ratio of IgE to non-IgE antibody or IgE to all epitope-specific antibody as a clinically-discriminating index of disease presence.

Suitable peptides are selected from the group consisting of:

| | |
|---|---|
| AAMEL; | (SEQ ID NO: 1) |
| ADARM; | (SEQ ID NO: 2) |
| AHKGF; | (SEQ ID NO: 3) |
| AHRET; | (SEQ ID NO: 4) |
| CDHKQ; | (SEQ ID NO: 5) |
| HRTFE; | (SEQ ID NO: 6) |
| HSYQE; | (SEQ ID NO: 7) |
| IPKQY; | (SEQ ID NO: 8) |
| KTGQFL; | (SEQ ID NO: 9) |
| LQTIQ; | (SEQ ID NO: 10) |
| PKNAW; | (SEQ ID NO: 11) |
| QAPEY; | (SEQ ID NO: 12) |
| RHVDCS; | (SEQ ID NO: 13) |
| SHHPA; | (SEQ ID NO: 14) |
| SPMAR; | (SEQ ID NO: 15) |
| TINSH; | (SEQ ID NO: 16) |
| TMDHAR; | (SEQ ID NO: 17) |
| VSKNML; | (SEQ ID NO: 18) |
| VTLRI; | (SEQ ID NO: 19) |
| WSCDH; | (SEQ ID NO: 20) |
| and | |
| YKSAH. | (SEQ ID NO: 21) |

An immune modulating molecular construct for a systemic adsorption of circulating antibodies includes:
a core hydrophilic molecule and covalently-coupled, 5-6 amino acid-length, epitope-mimicking peptides attached to the surface of the core hydrophilic molecule by way of an intervening soluble linker molecule. The linker also provides peptide solubility and projection away from the core protein and serves as the core hydrophilic molecule. The linker molecule may be polyethylene glycol.

Suitable peptides for the construct are selected from the group with the amino acid sequences:

| | |
|---|---|
| AAMEL; | (SEQ ID NO: 1) |
| ADARM; | (SEQ ID NO: 2) |
| AHKGF; | (SEQ ID NO: 3) |
| AHRET; | (SEQ ID NO: 4) |
| CDHKQ; | (SEQ ID NO: 5) |
| HRTFE; | (SEQ ID NO: 6) |
| HSYQE; | (SEQ ID NO: 7) |
| IPKQY; | (SEQ ID NO: 8) |
| KTGQFL; | (SEQ ID NO: 9) |
| LQTIQ; | (SEQ ID NO: 10) |
| PKNAW; | (SEQ ID NO: 11) |
| QAPEY; | (SEQ ID NO: 12) |
| RHVDCS; | (SEQ ID NO: 13) |
| SHHPA; | (SEQ ID NO: 14) |
| SPMAR; | (SEQ ID NO: 15) |
| TINSH; | (SEQ ID NO: 16) |
| TMDHAR; | (SEQ ID NO: 17) |
| VSKNML; | (SEQ ID NO: 18) |
| VTLRI; | (SEQ ID NO: 19) |
| WSCDH; | (SEQ ID NO: 20) |
| and | |
| YKSAH. | (SEQ ID NO: 21) |

The construct is useful for subcutaneous injection in a host to reduce an epitope-specific antibody or oral administration in a host to reduce an epitope-specific antibody.

An immunoassay which measures a quantitative relationship between harmful antibodies and protective antibodies includes:
quantifying epitope-specific harmful antibody isotypes;
quantifying epitope-specific protective antibody isotypes; and
calculating the ratio between harmful and protective antibody isotype specific for an individual epitope.

The immunoassay is useful to detect an autoimmune disease, wherein if the harmful antibody exceeds the ability of the protective antibody to block or moderate the binding of the harmful antibody, the disease is present.

Also disclosed is a plurality of protective humanized murine or other animal antibodies made by recombinant DNA techniques wherein the antibodies are characterized as:
capable of binding to the epitopes complexed by harmful antibodies; and
therapeutically administered in-vivo, to improve the balance between protective and harmful antibodies and/or immune cells to reduce or negate an autoimmune disease.

A therapeutic construct includes epitopic peptide sequences attached to a carrier molecule.

The therapeutic construct adsorbs harmful antibodies and/or stimulate production of protective antibodies, thereby favorably changing the balance between harmful and protective antibodies.

A harmful antibody may be defined for example as degranulating mast cells or fixing complement.

A competing antibody competes with harmful antibodies for binding sites.

### BRIEF DESCRIPTION OF THE DRAWINGS

FIG. 1 depicts a representation of the individual ratios of peptide-specific IgE to total peptide specific serum antibody where the individual peptides structurally and functionally mimic myelin protein-surface epitopes. (SEQ ID NOS 2, 1, 7, 12, 19, 4, 6, 9, 3, 10, 11, 14, 15, 21, 17, 5, 8, 16, 20, 13, 18 are disclosed respectively in order of appearance).

FIG. 2 depicts a representation of a therapeutic construct wherein the specific peptide needed to systemically adsorb harmful, autoimmune IgE is attached to the surface of a carrier protein represented by ragweed Ra5 allergenic protein.

FIG. 3 depicts the linear structure of the RaS protein and the amino acids likely to be on the protein surface highlighted in grey. The surface lysines (K) are the attachment points for the ADOOA molecule on the distal end of the ADOOA-ADOOA-peptide construct (SEQ ID NO: 22).

FIG. 4 depicts a representation of a therapeutic construct wherein the specific peptide needed to systemically adsorb harmful, autoimmune IgE is attached to an ADOOA doublet which confers the required aqueous solubility.

FIG. 5 depicts a representation of a therapeutic construct where the specific peptide needed to systemically adsorb harmful, autoimmune antibody has an attached hydrophilic amino acid (filled in circle) at either end so as to provide adequate aqueous solubility for the relatively hydrophobic peptide.

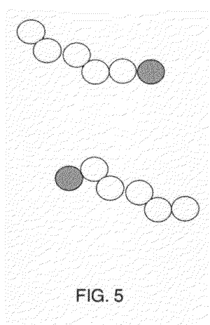

### DETAILED DESCRIPTION

Interactions between myelin-specific serum IgE, likely a harmful autoantibody, and corresponding, and competing, non-IgE antibodies, each targeting, in parallel, myelin autoimmune epitopes, are disclosed.

The inventor has found that utilization of pentameric or hexameric peptides as source antigens in quantifying serum IgE, alone, has yielded sensitivity of MS detection that approximates 60 percent. Factoring in the absence of corresponding, peptide-specific IgA, IgG, or IgM using and early-generation immunoassay system achieves an increase in sensitivity by only five to ten percent.

A new assay system identified the presence of multiple sclerosis (MS) by quantifying myelin autoimmune epitope-specific IgE and corresponding and competing non-IgE antibodies in patient sera. Patients with either (1) relapsing, remitting MS, (2) with secondary progressive MS, and (3) with primary progressive MS were tested. Test results were generated by quantifying the ratio of IgE/non-IgE epitope-specific antibody corresponding to twenty-one myelin-derived, single antibody, humoral epitopes. Five epitopes were located on the outer surface of myelin: one corresponding to proteolipid protein (PLP) and four corresponding to extracellular myelin oligodendrocyte glycoprotein (MOG). Ten autoimmune epitopes were located within sub-membrane strata of packed myelin made accessible by disease-effected myelin disruption or cyclical myelin reconstitution: one corresponding to Claudin 11, seven to myelin basic protein (MBP), and two to the intracellular domain of MOG. Six target epitopes corresponded to oligodendrocyte myelin glycoprotein, a protein located on the inner, axon-abutting, myelin surface and likely to be exposed by myelin disruption and reconstitution.

Peptides functionally identical to individual epitopes were used as source antigens to quantify epitope-specific serum IgE and total anti-myelin epitope-specific antibody. The latter was affected by measuring kappa plus lambda chain specific antibodies. The in-vitro tests employed were modified versions of the RAST test method known to those of skill in the art. Test results generated by using outer myelin surface peptides showed 83 percent sensitivity. Test results generated by using intra-myelin peptides showed 67 percent sensitivity. Test results generated by using periaxonal myelin peptides showed 33 percent sensitivity. Combined use of all myelin epitope-corresponding peptide antigens provided 100 percent sensitivity and specificity.

Potentially unique peptide sequences to use as source antigens were identified by searching the NIH protein sequence data base and discriminating 5-6 amino acid-length sequences found on the surface of myelin proteins but absent on the surfaces of other human proteins.

Recent technical observations have added two additional elements to understanding what constitutes unique peptide sequences: (1) presence of two or more individually hydrophobic, constituent amino acids per pentamer or hexamer and (2) display of a net hydrophilicity values of 4.8 or less (Hopp and Woods, 1981).

To date, conventional reasoning had been applied as to the major histocompatability complex (MHC) restrictions regulating epitope-specific antibody production and isotype selection. All or nothing limitations were expected as to epitope-specific antibody production, i.e., some people possess the correct MHC antigen-presenting capability and are able to produce antibodies against predetermined humoral epitopes, but others lack the correct MHC molecular machinery and therefore cannot. Members of the latter group were believed to produce antibodies against alternate epitopes under correspondingly different MHC regulation. Furthermore, epitope-specific antibody production was expected to favor a single antibody isotype, be it harmful or benign. Multiple, coexisting, epitope-specific antibody isotypes operating within an autoimmune process were not expected by earlier reasoning. The results disclosed herein were, therefore, surprising and unexpected.

MS Screening and Confirmation Test

Testing for the presence of serum IgE antibodies specific for synthetic peptides that are immunologically functionally equivalent to autoimmune epitopes such as those associated with multiple sclerosis (MS) offers a unique opportunity for diagnosis of autoimmune diseases. Such peptides also serve as components in novel therapeutics. Conventional wisdom holds that all immunoglobulins can be potentially harmful, but, as described herein, it is the quantitative difference between damaging antibody (i.e. IgE) and competing, non-toxic or protective antibody isotypes that predicates disease. Levels of both harmful and protective isotypes are considered for diagnosis and therapy of autoimmune diseases.

Autoimmune disease-affected individuals have relatively greater amounts of epitope-specific harmful antibodies (IgE, IgA₂, IgG₁, IgG₃, complement-fixing IgM) compared to levels of blocking antibodies (IgA₁, IgG2, IgG₄, and non-complement fixing IgM).

Functional, humoral autoimmune epitopes associated with multiple sclerosis (MS) were identified. Early stage MS patients were identified and differentiated from patients with other neurological conditions that are characterized by similar symptoms. An MS screening test is based upon multi-isotype, epitope-specific serum autoantibody quantification.

Twenty-one humoral epitopes have been identified on myelin proteins that are structurally and functionally unique. Inspection of the linear protein structure of individual myelin proteins was used to identify 5-6 amino acids in length, surface peptide regions that are candidates for autoimmune, antibody-reactive epitopes because these were functionally hydrophilic and exhibited unique amino acid sequences. For example, MS-specific, myelin-protein epitopes were identified through the use of molecular modeling software based upon the rolling sum analysis of 7 consecutive residues method developed by Hopp and Woods (1981). Highly sensitive and specific serum IgE and kappa plus lambda chain radio-immunoassays were employed to confirm specific peptide reactivity. The kappa plus lambda radioimmunoassay measures antibodies of all isotypes, IgE included. Because IgE usually represents less than 1/300,000 of total measured antibody in a serum sample, specific kappa plus lambda assay results based upon use of a 1/300 diluted serum can be used to reliably estimate all non-IgE specific antibodies.

Synthetic peptides serving as source antigens were then tested for immunoreactivity. The testing routine sequentially encompassed: (1) measuring specific serum IgE against each peptide antigen using a highly sensitive and specific immunoassay based upon a RAST format (Ceska, 1972); (2) and parallel testing of serum samples for the concomitant presence of competing peptide-specific non-IgE antibody (IgA and/or IgG and/or IgM quantified by a peptide-specific kappa chain plus lambda chain immunoassay); (3) dividing the IgE (harmful) antibody level by the competing antibody level in order to derive an effective IgE quotient value; (4) comparing the peptide-specific quotient value of a test subject against an established, peptide-specific quotient value scale for a specific disease or condition; and (5) assigning a positive or negative test result based upon the test subject quotient value being higher or lower than the threshold value of the scale, the scale having been established by comparing clinically-effected subjects and unaffected control subjects.

The sensitivity and specificity of the MS screening product in examples described herein exceed 90% based upon the use of Claudin 11 (OSP), myelin basic protein (MBP), myelin oligodendrocyte glycoprotein (MOG), oligodendrocyte myelin glycoprotein (OMgp), and proteolipid protein (PLP) peptide antigen-specific IgE quantification, and concomitant quantification of IgA and/or IgG and/or IgM blocking antibodies.

Five validated, MS-specific epitopes [Table 1] are located on the outer surface of the following myelin proteins: MOG and PLP. Ten validated, MS-specific epitopes are located on the outer surface of the following intra-myelin proteins: OSP, MBP, and MOG. Six validated, MS-specific epitopes are located on the periaxonal myelin surface protein OMgp. The humoral epitopic regions are 5-6 amino acids in length, the size preferred for a single epitope-specific antibody to bind.

An epitope-specific antibody is a physiologically protective or insulating antibody in so far as it successfully competes for specific epitope binding against the damaging antibodies, or blocks epitope binding by harmful or tissue-damaging lymphocytes, and is selected from the group consisting of
IgA 1 antibody;
IgG 2 antibody;
IgG 4 antibody;
IgM antibody that does not fix complement; and
IgE antibody that does not degranulate mast cells because it is not presented to mast cell membranes in a proper dimeric form.

MS patients possess deleterious myelin epitope-specific IgE antibodies but lack sufficient quantities of matching specific IgA, IgG, and/or IgM so as to block the potentially harmful effect of their IgE. In contrast, gender and age-matched control subjects who are IgE positive also possess sufficient quantities of matching specific IgA and/or IgG and/or IgM blocking antibodies.

Peptides that suitably mimic humoral epitopes on the surface of myelin and adjoined oligodendrocytes are: PLP-situated ADARM (SEQ ID NO: 2) and MOG-situated AAMEL (SEQ ID NO: 1), HSYQE (SEQ ID NO: 7), QAPEY (SEQ ID NO: 12), and VTLRI (SEQ ID NO: 19). Peptides that suitably mimic humoral epitopes on proteins located within packed myelin are: Claudin 11 (OSP) situated AHRET (SEQ ID NO: 4); MBP-situated AHKGF (SEQ ID NO: 3), LQTIQ (SEQ ID NO: 10), PKNAW (SEQ ID NO: 11), SHHPA (SEQ ID NO: 14), SPMAR (SEQ ID NO: 15), TMDHAR (SEQ ID NO: 17), and YKSAH (SEQ ID NO: 21); and intracellular MOG-situated HRTFE (SEQ ID NO: 6) and KTGQFL (SEQ ID NO: 9). Peptides that suitably mimic humoral epitopes on proteins located on the inner, periaxonal myelin surface are the OMgp-situated CDHKQ (SEQ ID NO: 5), IPKQY (SEQ ID NO: 8), TINSH (SEQ ID NO: 16), WSCDH (SEQ ID NO: 20), RHVDCS (SEQ ID NO: 13), and VSKNML (SEQ ID NO: 18).

A ratio of IgE to non-IgE antibody is a clinically-discriminating index of disease presence and which epitopes are being targeted in the disease process.

Autoimmune Disease Therapy

A therapeutic, immune modulating molecular construct suitable for injection consists of a 5-6 amino acid length, epitope-mimicking peptide attached to a ragweed allergen or other soluble carrier protein molecule via a polyethylene glycol (PEG) linker molecule. A shortened construct only comprising a specific peptide attached to a short PEG molecule is suitable for oral administration.

The constructs can be used therapeutically for systemic adsorption of circulating antibodies if the antibodies are having a deleterious effect upon the health of a treated subject.

Suitable peptides include those with the amino acid sequences in Table 1. To administer the therapeutic construct, one must: (a) Identify against which myelin protein epitopes, specific IgE antibodies exist in a patient's systemic circulation with insufficient quantity of specific non-IgE antibodies so as to block the harmful effect of the IgE; (b) Select a therapeutic construct that contains matching peptides to reduce or eliminate the IgE or other harmful antibody isotypes thereby ameliorating the adverse condition.

Subcutaneous injection or adequate oral administration of the therapeutic construct in a host achieves reduction in a particular epitope-specific antibody isotype. In MS, the therapeutic construct complexes with circulating specific IgE and non-IgE antibodies, thereby lowering both, most importantly, the specific IgE. Lowering or eliminating IgE slows down or stops the disease process.

Diagnostic peptides for autoimmune diseases are made as follows (See also Materials and Methods). A synthetic peptide of 5-6 amino acids in length adequately represents a two and three dimensional humoral epitope because its small size and freedom to bend allows it to precisely fit into the antigen binding site of a specific antibody if given enough time. The amino acid sequence structure of such a short peptide must be unique. The structural characteristics are: (a) location on the outer surface of the specific antigenic protein; (b) constituent presence within the peptide of two or more individually hydrophobic amino acids; and (c) a functional net hydrophilicity that does not exceed 4.8 by the Hopp and Woods method (1981). Site-specific peptides longer than 5-6 amino acids provide diminished specificity unless these are individually composed of overlapping, singularly unique pentameric sequences. Peptides shorter than 5 amino acids provide both diminished specificity and sensitivity.

Humoral two-dimensional (2D) epitopes are those that are 5-6 amino acids in length and closely represent sections of the linear contour of an amino acid chain.

In contrast, 3D epitopes are those antibody binding sites represented by radial molecular contours emanating from several adjoining linear contours and intervening spaces. A 3D contour is semi-perpendicular to its constituent 2D parallel contours. A 3D epitope-can be mimicked by a pentameric peptide whose contiguous amino acid sequence linearly represents the amino acids emanating from each 2D contour contact point and intervening spaces.

2D epitopes may be the principal immunogenic sites involved in instigating and sustaining a cellular or humoral immune process as these are likely to be more structurally stable and available than 3D epitopes whose conformations constantly changes because of the molecular flexing of their constituent, parallel contours.

Specific serum IgE antibody detection potentially ranges from 0.9 picogram/ml serum solution to 30 ng/ml serum solution. IgE is a principal pathological antibody isotype in MS because: (a) its functional in-vitro half-life is longer than other antibody isotypes; (2) in its conventional allergy role, IgE is commonly induced and sustained by numerous environmental antigens, many of which are encountered year-round; and (3) in its autoimmune role can be produced in response to epitopic mimicry of ingested or inhaled antigens (Table 2). The immunizing, affecting antigens can be found on environmental and mammal-colonizing microorganismal proteins and on proteins found on mold spore, and pollens. The antigens can also be found on the surface of proteins released from decaying environmental matter.

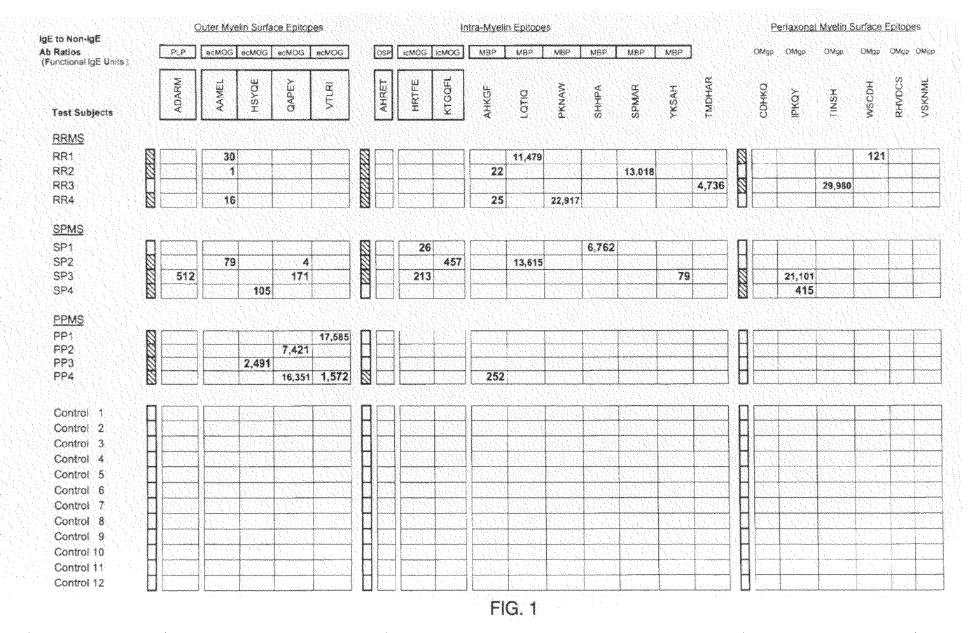

In quantitative order, the principal immunogenic, inhaled environmental factors are: (1) terrestrial bacterial proteins; (2) mold spore proteins; (3) pollen proteins; and (4) structural proteins of colonizing or infecting viruses. The key immunogenic elements on inhaled proteins are 5-6 amino acid-length peptide regions on the protein surface that structurally mimic unique sequences on human protein surfaces. These are identified by: (1) performing the sum-of-seven contiguous amino acids analysis method of Hopp and Woods to identify surface amino acid sequences of a target protein; (2) subdividing each surface sequence, left to right, into overlapping pentamers where the first sequential amino acid of the second, overlapping pentamer is the second amino acid of the first pentamer, etc.; (3) ascertaining whether the foreign protein possesses surface pentamers that are an exact match to a singular pentameric sequences on a human protein; and (4) determining whether some of the foreign protein pentamers overlap so as to yield matching hexamers. The 21 myelin protein peptide sequences displayed in FIG. 1 is unique in-so-far that each has no exact match on the surface of any other human protein but matches environmental protein surface pentamers.

FIG. 1 depicts a representation of the individual ratios of peptide-specific IgE to total peptide specific serum antibody where the individual peptides structurally and functionally mimic myelin protein-surface epitopes. (SEQ ID NOS 2, 1, 7, 12, 19, 4, 6, 9, 3, 10, 11, 14, 15, 21, 17, 5, 8, 16, 20, 13, 18 are disclosed respectively in order of appearance).

Both Specific IgE and total specific antibody values were ascertained by testing subject serum using a modified version of the radioallergosorbent test (Ceska) where a complex protein allergen mixture normally used to covalently couple to the paper discs was substituted by antigenic peptide constructs comprised of pentameric or hexameric peptides attached to an 8-Fmoc-amino-3, 6-dioxa-octanoic acid (ADOOA) linker/extender molecule.

Specific test background was determined by using paper discs that had only attached ADOOA linker/extender. Net epitope-specific IgE and net epitope-specific total antibody was ascertained by subtracting background values from corresponding peptide/ADOOA construct-specific values.

Individual IgE/total specific antibody ratios were processed by: (1) ascertaining peptide-specific IgE/total antibody ratios for disease-free subjects; (2) determining the control mean+x standard deviations where x varied from one epitopic peptide to the next so as to encompass all control subject results; (3) subtracting the derived peptide-specific control values from all subsequent test subject results; and (4) multiplying the net ratios by 1,000,000 in order to attain whole working numbers, termed Functional IgE Units (FIU).

Net positive FIU values are characteristic of MS disease-presence whereas zero or net negative FIU values indicate disease absence.

Significantly positive FIU values corresponding to outer myelin surface epitopes are likely indicators of secondary progressive (SP) MS or primary progressive (PP) MS, the severity of which linearly corresponds to the FIU value.

Significantly positive FIU values corresponding to surface epitopes of proteins buried within myelin or located on its periaxonal surface are indicators of relapsing remitting (RR) MS of secondary progressive MS.

Together with the correct MHC processing of antigen (genetic predisposition), all other elements needed for autoimmune disease initiation and sustenance operate in MS: (1) immunization with an inhaled foreign protein; (2) a break in tolerance occurring secondary to the single-site uniqueness of the myelin epitope; (3) chronic or extended exposure of the driving antigen(s); and (4) immune production of a harmful, epitope-specific antibody that is unaccompanied by a sufficient quantity of competing, blocking (neutralizing) antibody.

Using an ultrasensitive assay of high specificity shows: (1) relatively unencumbered (higher ratio of IgE to non-IgE antibody) myelin protein-specific IgE selectively present in MS patient sera against 4 distinct, structurally unique binding sites on the extracellular domain of myelin oligodendrocyte glycoprotein (MOG) and a single site on proteolipid protein (PLP); (2) the sites of IgE binding are sterically positioned to afford correct Fc projection for mast cell degranulation; and (3) the protein regions complexed are on the myelin exterior for favorable mast cell access. Structurally unique humoral binding sites on three other myelin proteins, Claudin 11, myelin basic protein (MBP), and the MOG intracellular domain become pathologically accessible once myelin outer cell layer has been breached. Among MS patients, site-specific IgE is relatively unencumbered because, unlike in control subjects, MS patients possess less competing, epitope-specific, non-IgE antibody.

The MS-specific serum test confirms symptomatic multiple sclerosis (MS) and identifies early, clinically silent MS. The basic test format involves simultaneous testing of subject sera for the presence of epitope-specific serum IgE and epitope-specific serum non-IgE antibodies. The test is an immunoassay wherein serum autoantibodies complex with peptides structurally mimicking humoral, autoimmune epitopes on myelin proteins. Each peptide: (1) is five to six amino acids long; (2) structurally and functionally mimics the surface region of its parent protein; and (3) offers a correct fit for the antigen binding site of a single specific autoantibody.

Of the tested tandem of specific IgE and non-IgE antibodies, IgE is the harmful, disease-effecting isotype through its induction of mast cell degranulation. A small quantity of correctly-positioned IgE elicits enormous damage by the amplifying effect of degranulation. Non-IgE antibodies serve as blocking antibodies that limit or prevent IgE binding and subsequent harmful effect. MS patients display relatively unencumbered IgE while IgE-positive controls possess significant quantities of non-IgE blocking antibody.

Compositions disclosed show diagnostic sensitivity and specificity that exceed 90% when the interactive effect of IgE relative to other, epitope-competing isotypes is measured.

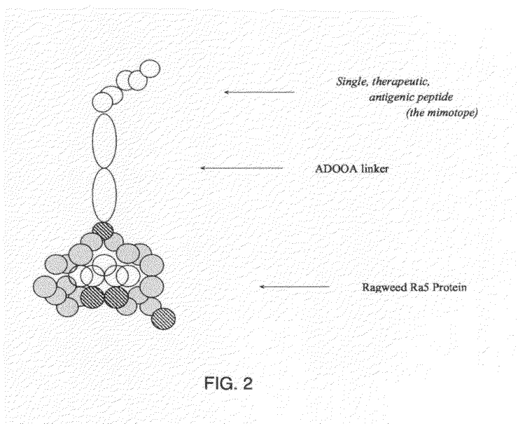

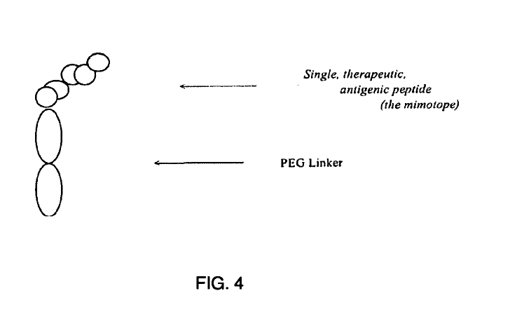

Construction of therapeutic neo-antigen molecules is disclosed for reduction or abrogation of the IgE-mediated, MS-specific inflammatory process. Elimination of harmful antibodies is directed to healing of active neural lesions (plaques) and prevention of new lesion formation. Basic structure of each molecule includes a core, soluble molecule such as the protein, ragweed Ra5, which has been in approved pharmaceutical use for sixty years, plus one attached 5-6 amino acid-length peptide. The peptide is connected to the core protein by a polyethylene glycol (PEG) linker (FIG. 4) or like molecule (FIG. 2) in order to provide solubility and molecular extension. The linker, alone, can serve as the soluble core molecule. Up to twenty-one neo-antigen molecules are formulated, each corresponding to a distinct autoimmune, epitope on a myelin protein (Table 1). Therapeutic administration simulates allergy desensitization (epitope-specific IgE quantification for initial dose estimation followed by graded dose increases and stabilization). Therapeutic outcomes are judged on the basis of lesion resolution and new lesion prevention as depicted by MRI. The basic tasks involve in therapeutic product introduction are: (1) Ra5 purification from ragweed pollen; (2) synthesis of individual PEG-peptide molecules; (3) conjugation of individual PEG-peptides to Ra5 carrier; (4) bottling of 21 individual pharmaceutical constructs; (5) an early-stage animal toxicity study to illustrate product safety; and (6) MS patient clinical trials.

Clinical test data indicates that anti-myelin IgE, in the absence of a sufficient quantity of blocking/interfering non-IgE antibody, is pathologically linked to multiple sclerosis. This was shown by increased specific IgE/blocking antibody ratios corresponding to increased disease severity among patients ranging from those with relapsing Remitting MS (milder form) to those with Primary Progressive MS (most severe form). The FIU against outer myelin surface epitopes averaged 31 for the relapsing remitting MS patients but increased 9-fold to 285 FIU among the patients with secondary progressive MS, and a 220-fold to 6895 FIU among patients with primary progressive MS.

A significant difference existed between relapsing remitting MS patient results and test results of the more severe groups with regard to intra-myelin epitope targeting. The relapsing remitting MS patients showed almost exclusive predilection toward intra-luminal myelin epitopes and frequently exhibited FIU values that were greater than those of the primary progressive patients shown against outer myelin surface epitopes.

The peptide-specific FIU results corresponding to humoral epitopic sites on oligodendrocyte myelin glycoprotein were equally representative of both patients and controls, suggesting that OMgp epitopic sites play a small role in MS pathology.

Utilization of test results based upon outer myelin surface epitopes provided a sensitivity of 83 percent and a specificity of 100 percent.

Employing test results only based upon intramyelin epitopes provided sensitivity of 42 percent.

Combining both myelin surface and intraluminal mimotopic peptide-specific test results provided combined ms diagnostic sensitivity and specificity of 100 percent.

Discussion

In the development of the methods and compositions described herein, first was the association between serum-borne, myelin-specific IgE and multiple sclerosis. Second was a likely explanation for disease causation and prolongation. Third was an explanation for why some MS patients experience a relatively mild and spontaneously reversible disease course (relapsing remitting MS) yet others develop a potentially lethal form (primary progressive MS). Fourth was recognition that the competing antibodies interplay is a likely hallmark of MS pathology and of autoimmune diseases pathology in general. Fifth was realization that a true, MS-specific therapeutic could be developed.

Detection of myelin-targeting antibodies in serum was important because it suggests that: (1) MS, an autoimmune disease, has its origins (IgE autoantibody production) systemically and not within the perineural space as was commonly believed; (2) systemic antibody production originates and is sustained by environmental antigenic stimuli; (3) many environmental stimuli, principally soil bacteria and other microorganisms, mold spores, pollens, and decaying organic matter, may exert an autoimmunizing and immune-boosting affect; (4) basic structural components of the immunizing materials are likely to be proteins or protein fragments that are inhaled; and (5) extracorporeal protein structures are ever-changing so as to eventually, mimic, on their respective surfaces, all human epitopic structures.

The number of potential humoral epitopes is not insignificant. Kabat's observation was confirmed by us that humoral epitopes are pentameric or hexameric in-size. The potential number of pentamers that are possible is 3.2 million and hexamers are about, 64 million. These numbers are probably too high to accurately describe a complete human autoimmune epitope library. Autoimmune, epitopic amino acid sequences display special structural characteristics having to do with inclusive hydrophobic amino acids and net hydrophilicity. Therefore, the potential number of pentameric/hexameric autoimmune structures in a human autoimmune epitope library may be greatly reduced to a much smaller number, approximately 228,000±152,000 component units. Therefore, the potential existence of a negative, environmental interaction in MS is highly likely, especially when one considers the ever-growing list of characterized microorganisms with MS-associated peptides already identified (Mikol, 2006) and the constant extracorporeal, microorganism turnover with its associated genetic drift that is certain to result in new effector strains.

After the correct pentameric or hexameric immunizing structure is provided by extracorporeal sources, the last barrier of defense of a mammal is epitope-specific isotype regulation, a genetically-controlled function. It is a complex, multivariate activity that is resistant to single factor interference by its reliance upon multiple isotype targeting of a single epitope. It is however incomplete resistance insofar as previous immunological studies have failed to consider that that some molecular surface, self-epitopes may occur but once in the entire human genome and therefore facilitate the breaking of self tolerance at those single points. The breaking of tolerance at those points would result in greater production of damaging antibody isotypes (i.e. IgE) or lesser production of blocking antibody isotypes thus causing focal tissue damage.

The immune-modulating affect of inhaled environmental immunogens upon relapsing remitting multiple sclerosis likely follows a predictable path: (1) the afflicted individual would have genetic susceptibility favoring production of specific IgE against environmental epitopes that structurally mimicked myelin basic protein, autoimmune epitopes; (2) the subject would be exposed to environmental immunogens that upregulate specific IgE production at the expense of competing non-IgE antibodies; (3) the target epitope(s) on MBP would be exposed by disruption of the outermost myelin membrane layer so as to provide functional access to roaming mast cells; and (4) mast cells would individually attach to two or more MBP-bound IgE antibodies and subsequently degranulate, releasing proteases and other myelin-destructive substances. Initial exposure of the myelin basic protein would be concurrent with the ongoing, homeostatic remodeling of individual, myelin compartments with loss of outer layer myelin membrane or would be induced by myelin toxic factors (i.e. vitamin B12 deficiency).

MS attacks may be brief for relapsing-remitting patients because of rapid myelin membrane repair resulting in functional withdrawal of myelin basic protein and its autoimmune epitopes. As relapsing remitting MS patients also appear to exhibit lower IgE/non-IgE antibody ratios against myelin surface epitopes, they are thus less likely to disrupt the membrane barrier that immunologically hides the MBP target antigen.

An opposing set of circumstances holds true for the more severe forms of MS: (1) focused production of IgE autoantibodies against myelin surface epitopes; (2) minimal or no pause in outer membrane destruction so as to permit myelin repair and reconstitution; (3) axonal exposure resulting in reduced nerve conduction; and (4) potential neuronal death.

A specific therapeutic solution is to formulate a molecular construct that adsorbs the autoimmune epitope-specific IgE and/or increases production of the IgE-blocking antibodies. Down-regulation or up-regulation of single-site-specific antibody production is a daunting technical challenge as practical, clinically oriented experience does not exist to either help develop the therapeutic construct nor its utilization. However, construction and clinical utilization of an IgE adsorptive molecule is feasible and predictable as it includes elements and practices that are currently in general use for allergy desensitization.

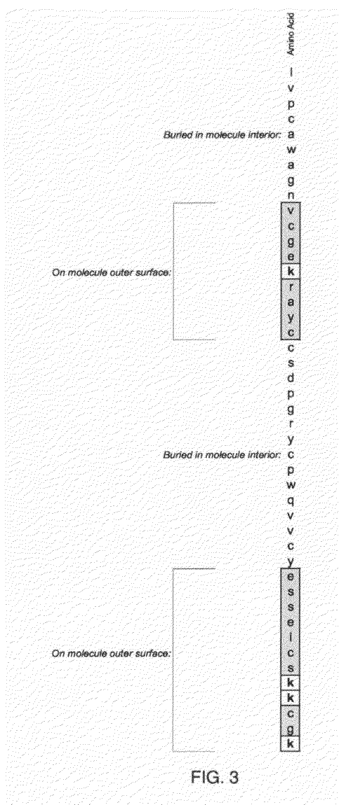

A therapeutic construct contemplated is illustrated in FIG. 3. It consists of a core carrier protein, ragweed allergen Ra5, and a single myelin autoimmune mimotopic peptide attached to the carrier protein by a PEG linker. Another contemplated therapeutic construct comprises the specific peptide attached to the PEG linker. PEG, already in general use as a pharmaceutical additive, provides peptide solubility. When attached to Ra5, it provides extension away from the ragweed protein so as to be easily accessible for binding by anti-myelin, epitope-specific autoantibodies. A single peptide is conjugated per carrier protein so as to prevent dimerization between the therapeutic molecule and targeting specific IgE antibodies.

The binding of a single epitope per IgE molecule should be sufficient to alter the 3D structure of the IgE molecule so as to make it recognizable for reticulo-endothelial clearance. The therapeutic neomolecules are handled in a similar fashion to clinical utilization of extracted allergenic proteins: (1) quantification of construct-specific serum IgE via a modified RAST analysis; (2) utilization of the RAST score to determine a starting subcutaneous injection dose or oral starting dose; (3) sequential increases in interval dosage; and (4) monitoring of therapeutic efficacy through sequential physical examination and interval MRI examination. Also likely will be use of the MS screening test to quantify a sequential reduction in starting FIU level(s) post treatment.

Peptides consisting essentially of the epitopes or any other specific regions of a protein include amino acids from the target peptide and any other amino acids that do not substantially

### Materials and Methods

### Peptide-Specific IgE Measurement

### Quantification of Epitope-Specific Serum IgE

(1) Paper discs obtained from Schleicher and Schuell (Keene, N.H.) are cyanogen bromide-activated (Ceska et al., 1972), and then separately conjugated with 4 nanomoles of individual lysine-PEG linker-peptides (Mimotopes, Clayton, Australia) diluted in 20 mM phosphate buffered saline, pH 7.3 (PBS) and 4 nanomoles of lysine-PEG linker ((Mimotopes, Clayton, Australia) diluted in PBS.
(2) In order to measure mimotopic peptide-specific serum IgE, care is taken to avoid potential interference from anti-PEG antibodies and anti-cellulose antibodies. Toward that end, individual serum samples (1 ml each) are incubated for 24 to 72 hours with two lysine-PEG linker-conjugated discs and two unconjugated paper discs to adsorb all specific background IgE in the sample.
(3) The paper discs are moved from the serum sample.
(4) 100 uL neat, adsorbed test subject serum is added to individual, peptide-conjugated paper discs and blank (background) discs in quadruplicate.
(5) Serum and discs are incubated for twenty hours at 37° C.
(6) Post-incubation, discs were aspirated and washed ×4 with PBST wash buffer (20 mM PBS plus 0.01% Tween₂₀).
(7) Fifty microliters I¹²⁵-labeled, affinity-purified, polyclonal goat anti-IgE (Vector Laboratories, Burlingame, Calif. USA; 40,000 counts/min diluted in 2% heat treated horse serum) is individually applied to the discs, followed by incubation at 37° C. for 20 hrs.
(8) Post-incubation, the discs are aspirated, washed ×4 with PBST, and counted in a Perkin Elmer Cobra gamma counter (Meridien, Conn. USA).
   Quantification of Peptide-Specific kappa+lambda Light Chain-Containing Antibody (Total Epitope-Specific Antibody).
(1) Paper discs obtained from Schleicher and Schuell (Keene, N.H.) are cyanogen bromide-activated (Ceska et al., 1972), and then separately conjugated with 1 nanomole of individual lysine-PEG linker-peptides (Mimotopes, Clayton, Australia) diluted in 20 mM phosphate buffered saline, pH 7.3 (PBS) and 1 nanomole of lysine-PEG linker ((Mimotopes, Clayton, Australia) diluted in PBS.
(2) In order to measure mimotopic peptide-specific serum IgE, care is taken to avoid potential interference from anti-PEG antibodies and anti-cellulose antibodies. Toward that end, individual serum samples (1 ml each) are incubated for 24 to 72 hours with two lysine-PEG linker-conjugated discs and two unconjugated paper discs to adsorb all specific background IgE in the sample.
(3) The paper discs are moved from the serum sample.
(4) The serum sample is diluted 1:300 with assay wash buffer (PBST).
(5) 100 uL diluted, adsorbed test subject serum is added to individual, peptide-conjugated paper discs and blank (background) discs in quadruplicate.
(6) Serum and discs are incubated for twenty hours at 37° C.
(7) Post-incubation, discs are aspirated and washed ×4 with PBST wash buffer (20 mM PBS plus 0.01% Tween₂₀).
(8) Fifty microliters I¹²⁵-labeled, affinity-purified, polyclonal goat anti-(kappa+lambda) light chain antibody (Vector Laboratories, Burlingame, Calif. USA; 40,000 counts/min diluted in 2% heat treated horse serum) is individually applied to the discs, followed by incubation at 37° C. for 20 hrs.
(8) Post-incubation, the discs are aspirated, washed, and counted in a Perkin Elmer Cobra gamma counter (Meridien, Conn. USA).

### Molecular Structure of the Allergen/Peptide MS-specific Therapeutic.

Each Ra5 epitope-specific neomolecule includes a core protein and a covalently attached monovalent short peptide (5-6 contiguous amino acids) much like a bottle brush where the core protein is wire onto which is attached a single bristle, the specific peptide.

The starting core protein is the ragweed Ra5 antigenic protein or a similar protein that has been proved safe an effective in prior therapeutic products.

To administer the therapeutic product, initial injections preferably commence in a healthcare setting. After 3-4 serially incremental injections, the patient or home-based caregiver can administer the weekly maintenance injections. Therapeutic outcome is judged on MS symptom reduction, reduction of the number of Gd+ enhancing or new T2 lesions on MRI, and on reduction in MS relapses.

### Administration of the PEG/Peptide, MS-specific Therapeutic.

The product is administered orally, encapsulated to bypass gastric and duodenal degradation.

Initial administration commence in a healthcare setting. After 3-4 serially incremental oral doses, the patient can consume the peptide construct-filled capsule(s) at home. Therapeutic outcome is judged on MS symptom reduction, reduction of the number of Gd+ enhancing or new T2 lesions on MRI, and on reduction in MS relapses.

### Materials and Assay Preparation.

Paper discs obtained from Schleicher and Schuell (Keene, N.H.) were cyanogen bromide-activated (Ceska, 1972), and then separately conjugated with individual peptide constructs each comprising a specific pentamer or hexamer with a solublizing/projecting 8-amino-3,6-dicyclohexylidine-3-ethylbutyl group (PEG) linker (Mimotopes, Clayton, Australia). The non-peptide end of the linker contained a free amino group useful for covalent CNBr coupling. Approximately 10 or 40 nanomoles peptide construct in 20 mM PBS, pH 7.4 was applied per activated paper disc. Also conjugated to separate paper discs was equal molar quantity of aminated PEG for use in reducing background.

### Data Reduction.

Because of the unpredictability in removing all anti-cellulose and anti-PEG serum antibodies and the need to measure very low levels of humoral, epitope-specific IgE and specific non-IgE antibody, the data reduction method is configured to be impervious to small remaining quantities of each after serum scrubbing. This is done by estimating specific IgE and total specific antibody test background levels in order that these can be respectively subtracted from the specific IgE and total specific antibody results, thereby deriving net specific IgE and total specific antibody levels.

Quantification of harmful antibody (IgE) is relative to coexisting and competing/blocking non-IgE antibodies. As the specific IgE presence in serum is estimated to be at least 1/300,000 that of non-IgE antibody, total measured antibody, represented by the kappa plus lambda light chain value, was used to represent epitope-specific, non-IgE antibody. Counted paper disc values for specific IgE determination and kappa plus lambda chain determination were processed by: (1) averaging the middle two values of each quadruplicate point set; (2) determining which of 21 averaged values was the lowest; (3) multiplying the lowest value by 99.9 percent to derive the estimated working background value; and (4) subtracting the individually-derived background from each of the 21 averaged, peptide-specific antibody values in order to estimate the relative amount of net peptide-specific IgE relative to the total peptide-specific antibody.

In order to quantify the percent relationship between epitope-specific IgE and corresponding total antibody in a serum sample, the peptide-specific IgE result was divided by its corresponding light chain-specific antibody result and calling the ensuing value the “IgE quotient value” (QV). Peptide-specific quotient values are generated for all peptide-specific test results per tested serum sample.

Next, the mean and standard deviation (SD) of all peptide-specific quotient values per peptide-specific test are ascertained for a numerically significant, age-significant, and gender-significant control subject group. A specific threshold SD is calculated for each mimotopic peptide-specific test in order to encompass all control quotient values, peptide by peptide. These are the mimotopic peptide-specific threshold values. The mimotopic peptide-specific threshold values are multiplied by 1,000,000 in order to attain whole, not decimal, numerical working values called functional IgE units (FIU).

When testing subject serum for the MS presence, the peptide-specific threshold FIU is subtracted from the subject's corresponding test signal. If the net FIU result is a positive value, the test subject has MS. If the corresponding epitopes are located on the outer myelin surface, the MS category is likely to be secondary progressive MS or primary progressive MS. If the corresponding epitopes are located on the periaxonal surface of myelin, the MS category is likely to be primary progressive MS.

TABLE 1

Myelin outer surface epitopes:
ADARM (SEQ ID NO: 2): (PLP).
AAMEL: (SEQ ID NO: 1) (MOG).
HSYQE (SEQ ID NO: 7): (MOG).
QAPEY (SEQ ID NO: 12): (MOG).
VTLRI (SEQ ID NO: 19): (MOG).
Intra-myelin epitopes:
AHRET (SEQ ID NO: 4): (Claudin 11).
AHKGF (SEQ ID NO: 3): (MBP).
LQTIQ (SEQ ID NO: 10): (MBP).
PKNAW (SEQ ID NO: 11): (MBP).
SHHPA (SEQ ID NO: 14): (MBP).
SPMAR (SEQ ID NO: 15): (MBP).
YKSAH (SEQ ID NO: 21): (MBP).
TMDHAR (SEQ ID NO: 17): (MBP).
HRTFE (SEQ ID NO: 6): (MOG).
KTGQFL (SEQ ID NO: 9): (MOG).
Peri-axonal myelin surface epitopes:
CDHKQ (SEQ ID NO: 5): (OMgp).
IPKQY (SEQ ID NO: 8): (OMgp).
TINSH (SEQ ID NO: 16): (OMgp).
WSCDH (SEQ ID NO: 20): (OMgp).
RHVDCS (SEQ ID NO: 13): (OMgp).
VSKNML (SEQ ID NO: 18): (OMgp).

TABLE 2
Extraneous Protein Source

| | | | | | |
|---|---|---|---|---|---|
| Myelin | | | | | |
| Autoimmune | Bac- | | In- | | |
| Epitopes: | teria | Fungi | sects | Plants | Viruses |

| | | | | | |
|---|---|---|---|---|---|
| AAMEL | 4 | 2 | 3 | 0 | 0 |
| (SEQ ID NO: 1) | | | | | |
| HSYQE | 1 | 0 | 1 | 0 | 0 |
| (SEQ ID NO: 7) | | | | | |
| QAPEY | 6 | 3 | 0 | 0 | 0 |
| (SEQ ID NO: 12) | | | | | |
| VTLRI | 15 | 0 | 1 | 0 | 0 |
| (SEQ ID NO: 19) | | | | | |
| ADARM | 20 | 5 | 1 | 1 | 2* |
| (SEQ ID NO: 2) | | | | | |

*Molluscum contagiosum Subtype 1
*Vaccinia

Table 1 depects twenty-one structurally unique myelin peptides corresponding to autoimmune epitopes and useful for construction of an MS serum diagnostic test and an MS therapeutic construct.

Table 2 illustrates a number of extraneous source proteins that possess surface peptide sequences mimicking myelin oligodendrocyte glycoprotein (MOG) and proteolipid protein (PLP) surface humoral epitopes.

### PUBLICATIONS

The following are incorporated by reference to the extent they relate materials and methods of the present disclosure.
Ceska, M., Eriksson, R., Varga, J. M., 1972. Radioimmunosorbent assay of allergens. J. Allergy Clin. Immunol. 49, 1-9.
Hopp T P and Woods K T (1981) Prediction of protein antigenic determinants from amino acid sequences. Proc. Nati. Acad. Sci. USA Vol. 78, No. 6, pp. 3824-3828.

1. A plurality of target immunogenic peptides of a target protein wherein the target peptides produce a disease-specific immune response in a host, and wherein the target protein is causative of or associated with, a targeted disease, and wherein the peptides comprise the following structure:
(a) are 5-6 amino acids in length;
(b) have an amino acid sequence which is identical to a contiguous amino acid peptide region of a sequence of a protein designated the target protein;
(c) are characterized by a net hydrophilic structure, wherein the peptide is present while on the protein surface;
(d) include two or more individually hydrophobic, constituent amino acids;
(e) display a net hydrophilicity value of about 4.8 or less, wherein the peptide is analyzed separately from its parent protein sequence; and
(f) are individually characterized by an amino acid sequence that is structurally unique as to its amino acid sequence order.

2. The plurality of target immunogenic peptides of claim 1 further characterized as having antigenic profiles which elicit an immune response specific for the target protein as determined by results of immunoassays of disease positive biological fluids compared to disease negative biological fluids.

3. A method for diagnosing a disease in a subject wherein a peptide is used as a source antigen to quantify epitope-specific, potentially harmful antibody levels and levels of competing antibodies in a biological fluid, for an associated disease the method comprising:
(a) dividing the harmful antibody levels by the level of the competing antibodies to derive a quotient value;
(b) comparing the quotient value of the subject to a quotient value range predetermined for a specific disease; and
(c) diagnosing the disease by assigning a positive or negative test result based upon the subject's quotient value being higher or lower than a disease-positive threshold quotient value.

4. The method of claim 3 wherein the harmful antibody is an IgE antibody.

5. The method of claim 3 wherein the harmful antibody is an IgA₂, IgG₁, IgG₃, or complement-fixing IgM antibody.

6. The method of claim 3 wherein the harmful antibody is an opsonizing antibody.

7. The method of claim 3 wherein the competing antibody is an IgA₁, IgG₂, IgG₄, or non-complement-fixing IgM antibody.

8. A method to screen for multiple sclerosis, the method comprising:
(a) detecting myelin epitope-specific IgE antibodies to peptides serving as source antigens which structurally mimic humoral epitopes on the surface of myelin;
(b) ascertaining blocking antibodies by quantifying individual human kappa-chain-specific antibodies plus lambda chain-specific antibodies to the epitope-mimicking peptide and subtracting the quantity of the specific IgE, to reflect non-IgE specific antibody; and
(c) computing a ratio of IgE to non-IgE antibody or IgE to all epitope-specific antibody as an indicator of disease presence.

9. The method of claim 8 wherein the peptides are selected from the group consisting of:

| | | |
|---|---|---|
| (a) | AAMEL; | (SEQ ID NO: 1) |
| (b) | ADARM; | (SEQ ID NO: 2) |
| (c) | AHKGF; | (SEQ ID NO: 3) |
| (d) | AHRET; | (SEQ ID NO: 4) |
| (e) | CDHKQ; | (SEQ ID NO: 5) |
| (f) | HRTFE; | (SEQ ID NO: 6) |
| (g) | HSYQE; | (SEQ ID NO: 7) |
| (h) | IPKQY; | (SEQ ID NO: 8) |
| (i) | KTGQFL; | (SEQ ID NO: 9) |
| (j) | LQTIQ; | (SEQ ID NO: 10) |
| (k) | PKNAW; | (SEQ ID NO: 11) |
| (l) | QAPEY; | (SEQ ID NO: 12) |
| (m) | RHVDCS; | (SEQ ID NO: 13) |
| (n) | SHHPA; | (SEQ ID NO: 14) |
| (o) | SPMAR; | (SEQ ID NO: 15) |
| (p) | TINSH; | (SEQ ID NO: 16) |
| (q) | TMDHAR; | (SEQ ID NO: 17) |
| (r) | VSKNML; | (SEQ ID NO: 18) |
| (s) | VTLRI; | (SEQ ID NO: 19) |
| (t) | WSCDH; | (SEQ ID NO: 20) |
| and | | |
| (u) | YKSAH. | (SEQ ID NO: 21) |

10. An immunogenic modulating molecular construct for a systemic adsorption of circulating antibodies comprising:
(v) a core hydrophilic molecule; and
(w) covalently-coupled, 5-6 amino acid-length, epitope-mimicking peptides attached to the surface of the core hydrophilic molecule by way of an intervening soluble linker molecule, the linker also providing peptide solubility and projection away from the core molecule and separately able to serve as the core hydrophilic molecule.

11. The construct of claim 10 wherein the linker molecule is polyethylene glycol.

12. The construct of claim 10 wherein the linker molecule is a monomer or polymer of 8-Fmoc-amino-3, 6-dioxa-octanoic acid.

13. The construct of claim 10 wherein the peptides are selected from the group with the amino acid sequences:

| | | |
|---|---|---|
| (a) | AAMEL; | (SEQ ID NO: 1) |
| (b) | ADARM; | (SEQ ID NO: 2) |
| (c) | AHKGF; | (SEQ ID NO: 3) |
| (d) | AHRET; | (SEQ ID NO: 4) |
| (e) | CDHKQ; | (SEQ ID NO: 5) |
| (f) | HRTFE; | (SEQ ID NO: 6) |
| (g) | HSYQE; | (SEQ ID NO: 7) |
| (h) | IPKQY; | (SEQ ID NO: 8) |
| (i) | KTGQFL; | (SEQ ID NO: 9) |
| (j) | LQTIQ; | (SEQ ID NO: 10) |
| (k) | PKNAW; | (SEQ ID NO: 11) |
| (l) | QAPEY; | (SEQ ID NO: 12) |
| (m) | RHVDCS; | (SEQ ID NO: 13) |
| (n) | SHHPA; | (SEQ ID NO: 14) |
| (o) | SPMAR; | (SEQ ID NO: 15) |
| (p) | TINSH; | (SEQ ID NO: 16) |
| (q) | TMDHAR; | (SEQ ID NO: 17) |
| (r) | VSKNML; | (SEQ ID NO: 18) |
| (s) | VTLRI; | (SEQ ID NO: 19) |
| (t) | WSCDH; | (SEQ ID NO: 20) |
| and | | |
| (u) | YKSAH. | (SEQ ID NO: 21) |

14. Use of the construct of claim 10 to reduce an epitope-specific antibody in a biological fluid.

15. An immunoassay which measures a quantitative relationship between harmful antibodies and protective antibodies, the immunoassay comprising:
(v) quantifying epitope-specific harmful antibody isotypes;
(w) quantifying epitope-specific protective antibody isotypes; and
(x) calculating the ratio between harmful and protective antibody isotype specific for an individual epitope.

16. The immunoassay of claim 15 used to detect an autoimmune disease, wherein if the harmful antibody exceeds the ability of the protective antibody to block or moderate the binding of the harmful antibody, the disease is present.

17. A plurality of protective antibodies made by recombinant DNA techniques wherein the antibodies are characterized as:
(a) capable of binding to the epitopes complexed by harmful antibodies; and
(b) therapeutically administered in-vivo, to improve the balance between protective and harmful antibodies and/or immune cells to reduce or negate an autoimmune disease.

18. The antibodies of claim 17 are humanized.

19. A construct comprising epitopic peptide sequences attached to a carrier molecule.

20. The construct of claim 19 used to adsorb harmful antibodies and/or stimulate production of protective antibodies, thereby favorably changing the balance between harmful and protective antibodies.


SEQUENCE LISTING (22 sequences)

SEQ ID NO: 1 - protein, 5 aa, Artificial Sequence
  Description of Artificial Sequence Synthetic peptide
AAMEL

SEQ ID NO: 2 - protein, 5 aa, Artificial Sequence
  Description of Artificial Sequence Synthetic peptide
ADARM

SEQ ID NO: 3 - protein, 5 aa, Artificial Sequence
  Description of Artificial Sequence Synthetic peptide
AHKGF

SEQ ID NO: 4 - protein, 5 aa, Artificial Sequence
  Description of Artificial Sequence Synthetic peptide
AHRET

SEQ ID NO: 5 - protein, 5 aa, Artificial Sequence
  Description of Artificial Sequence Synthetic peptide
CDHKQ

SEQ ID NO: 6 - protein, 5 aa, Artificial Sequence
  Description of Artificial Sequence Synthetic peptide
HRTFE

SEQ ID NO: 7 - protein, 5 aa, Artificial Sequence
  Description of Artificial Sequence Synthetic peptide
HSYQE

SEQ ID NO: 8 - protein, 5 aa, Artificial Sequence
  Description of Artificial Sequence Synthetic peptide
IPKQY

SEQ ID NO: 9 - protein, 6 aa, Artificial Sequence
  Description of Artificial Sequence Synthetic peptide
KTGQFL

SEQ ID NO: 10 - protein, 5 aa, Artificial Sequence
  Description of Artificial Sequence Synthetic peptide
LQTIQ

SEQ ID NO: 11 - protein, 5 aa, Artificial Sequence
  Description of Artificial Sequence Synthetic peptide
PKNAW

SEQ ID NO: 12 - protein, 5 aa, Artificial Sequence
  Description of Artificial Sequence Synthetic peptide
QAPEY

SEQ ID NO: 13 - protein, 6 aa, Artificial Sequence
  Description of Artificial Sequence Synthetic peptide
RHVDCS

SEQ ID NO: 14 - protein, 5 aa, Artificial Sequence
  Description of Artificial Sequence Synthetic peptide
SHHPA

SEQ ID NO: 15 - protein, 5 aa, Artificial Sequence
  Description of Artificial Sequence Synthetic peptide
SPMAR

SEQ ID NO: 16 - protein, 5 aa, Artificial Sequence
  Description of Artificial Sequence Synthetic peptide
TINSH

SEQ ID NO: 17 - protein, 6 aa, Artificial Sequence
  Description of Artificial Sequence Synthetic peptide
TMDHAR

SEQ ID NO: 18 - protein, 6 aa, Artificial Sequence
  Description of Artificial Sequence Synthetic peptide
VSKNML

SEQ ID NO: 19 - protein, 5 aa, Artificial Sequence
  Description of Artificial Sequence Synthetic peptide
VTLRI

SEQ ID NO: 20 - protein, 5 aa, Artificial Sequence
  Description of Artificial Sequence Synthetic peptide
WSCDH

SEQ ID NO: 21 - protein, 5 aa, Artificial Sequence
  Description of Artificial Sequence Synthetic peptide
YKSAH

SEQ ID NO: 22 - protein, 44 aa, Artificial Sequence
  Description of Artificial Sequence Synthetic construct
VPCAWAGNVCGEKRAYCCSDPGRYCPWQVVCYESSEICSKKCGK